(19) United States
(12) Patent Application Publication
(10) Pub. No.: US 2026/0325098 A1
(43) Pub. Date: Oct. 22, 2026

(54) PHYSIOLOGICAL SOUND COLLECTION APPARATUS AND WEARABLE DEVICE

(71) Applicant: GOERTEK TECHNOLOGY CO., LTD, Qingdao, Shandong (CN)

(72) Inventors: Yong QIE, Qingdao, Shandong (CN); Maoqing TANG, Qingdao, Shandong (CN); Yongcheng ZHANG, Qingdao, Shandong (CN); Junjie PAN, Qingdao, Shandong (CN)

(21) Appl. No.: 19/111,383

(22) PCT Filed: Oct. 27, 2023
(86) PCT No.: PCT/CN2023/127153

§ 371 Date: Mar. 13, 2025

(30) Foreign Application Priority Data
Oct. 31, 2022 (CN) 202211351541.X

(51) Int. Cl. A61B 7/04; A61B 5/00; A61B 5/0205; A61B 5/021; A61B 5/11; H04R 1/08; H04R 1/10

(52) U.S. Cl. CPC A61B 7/04; A61B 5/0205; A61B 5/1118; A61B 5/4872; A61B 5/6802; A61B 5/6844; A61B 5/7278; H04R 1/08; H04R 1/10; A61B 5/021; A61B 2560/0247; H04R 2410/05; H04R 2420/07; H04R 2460/13

(57) ABSTRACT

Some embodiments of the present disclosure disclose a physiological sound collection apparatus and a wearable device, the physiological sound collection apparatus includes a bone conduction sensor and a first microphone, wherein the bone conduction sensor is configured to collect a physiological sound signal, the first microphone is configured to collect a first environmental noise signal, and the first environmental noise signal is used to correct the physiological sound signal. In the present disclosure, the physiological sound signal is collected by the bone conduction sensor in the physiological sound collection apparatus, so that the physiological sound can be collected by the user of the apparatus without the operation of the doctor. In addition, after the physiological sound signal is corrected by using the first environmental noise signal collected by the first microphone, accuracy of the physiological sound signal is improved.

The present application claims priority to a Chinese Patent Application No. 202211351541.X, filed on Oct. 31, 2022 to the Chinese Patent Office, with the invention name of “Physiological Sound Collection Apparatus and Wearable Device”, which is hereby incorporated by reference in its entirety.

### TECHNICAL FIELD

The present disclosure relates to a technical field of information collection, and in particular, to a physiological sound collection apparatus and a wearable device.

### BACKGROUND

Physiological sound refers to sound generated by mechanical wave phenomenon in human organs such as heart and lung.

At present, a manner of acquiring physiological sound is to perform auscultation when seeking medical attention, and the auscultation needs to be implemented by a doctor in a hospital, resulting in inconvenient collection of the physiological sound.

### SUMMARY

In view of this, the present disclosure provides a physiological sound collection apparatus and a wearable device, aiming to improve convenience of obtaining the physiological sound.

In order to achieve the above object, the present disclosure provides a physiological sound collection apparatus, including:
a bone conduction sensor and a first microphone,
wherein the bone conduction sensor is configured to collect a physiological sound signal, the first microphone is configured to collect a first environmental noise signal, and the first environmental noise signal is used to remove environmental noise characteristics from the physiological sound signal.

As an example, the apparatus further includes a second microphone, wherein the second microphone is configured to collect a second environmental noise signal; and the environmental noise characteristics are removed from the physiological sound signal only when the second environmental noise signal contains the same environmental noise characteristics as that contained in the first environmental noise signal.

As an example, the apparatus further includes a reminder module, wherein the reminder module is configured to output first reminder information to remind a user to adjust a measurement posture when a physiological sound signal intensity of the physiological sound signal is less than or equal to a preset physiological sound signal intensity threshold; and the reminder module is further configured to output second reminder information to remind the user to select a new environment to collect the physiological sound when a noise signal intensity of the second environmental noise signal is greater than or equal to a preset noise signal intensity threshold.

As an example, the wearable device includes a wearable body and an earphone, wherein the earphone includes the physiological sound collection apparatus described above.

As an example, the wearable body includes a first communication module, and the earphone includes a second communication module;
the first communication module is configured to receive, from the second communication module, the physiological sound signal from which the environmental noise characteristics have been removed;
the first communication module includes a Bluetooth module or a UWB module; and
the second communication module includes a Bluetooth module or a UWB module.

As an example, the wearable body further includes a heart monitoring trigger data collection module; the heart monitoring trigger data collection module is configured to collect heart monitoring trigger data; the heart monitoring trigger data is used to determine whether a user requires enhanced heart health monitoring, and when it is determined that the user requires enhanced heart health monitoring, the wearable body enters a heart monitoring mode.

As an example, the heart monitoring trigger data collection module is a body fat detection module, and the heart monitoring trigger data is a body fat rate; and if the body fat rate is greater than or equal to a preset body fat rate threshold, it is determined that the user requires enhanced heart health monitoring, and the wearable body enters the heart monitoring mode.

As an example, the heart monitoring trigger data collection module is a motion detection module, and the heart monitoring trigger data is motion data; the motion data is used to determine whether a frequency at which the user participates in exercise is less than or equal to a preset frequency threshold, and when it is determined that the frequency at which the user participates in exercise is less than or equal to the preset frequency threshold, it is determined that the user requires enhanced heart health monitoring, and the wearable body enters the heart monitoring mode.

As an example, the heart monitoring trigger data collection module includes a body fat detection module and a motion detection module, and the heart monitoring trigger data includes a body fat rate and motion data; if the body fat rate is greater than or equal to a preset body fat rate threshold and a frequency at which the user participates in exercise is less than or equal to a preset frequency threshold, it is determined that the user requires enhanced heart health monitoring, and the wearable body enters the heart monitoring mode.

As an example, the wearable body further includes a wearing detection module and a physiological parameter signal collection module;
the wearing detection module is configured to determine whether the wearable body is in a wearing state;
the physiological parameter signal collection module is configured to collect a physiological parameter signal of the user when the wearable body is in the wearing state.

As an example, the wearable body further includes a processor;
the processor is configured to calculate a blood pressure of the user based on the physiological sound signal from which the environmental noise characteristics have been removed and the physiological parameter signal, and perform a corresponding operation based on the calculated blood pressure.

Compared with a manner of acquiring physiological sound in which auscultation is performed when seeking medical attention, and the auscultation needs to be implemented by a doctor in a hospital, resulting in inconvenient collection of the physiological sound in the prior art, the present disclosure provides a physiological sound collection apparatus including a bone conduction sensor and a first microphone, wherein the bone conduction sensor is configured to collect a physiological sound signal, the first microphone is configured to collect a first environmental noise signal, and the first environmental noise signal is used to correct the physiological sound signal. In the present disclosure, the physiological sound signal is collected by the bone conduction sensor in the physiological sound collection apparatus, so that the physiological sound can be collected by the user of the apparatus without the operation of the doctor. In addition, after the physiological sound signal is corrected by using the first environmental noise signal collected by the first microphone, accuracy of the physiological sound signal is improved.

### BRIEF DESCRIPTION OF DRAWINGS

In order to more clearly illustrate the technical solution of the embodiments of the disclosure or the prior art, the drawings needed to be used by the embodiments or the prior art will be briefly described in the following. Obviously, the drawings in the following description are only related to some embodiments of the disclosure, and for those skilled in the art, other drawings may be obtained according to the structures illustrated in these drawings without creative efforts.

FIG. 1 is a schematic diagram of a physiological sound signal (upper) and a first environmental noise signal (lower) collected by a physiological sound collection apparatus of the present disclosure;

FIG. 2 is a schematic structural diagram of an earphone involved in the physiological sound collection apparatus according to an embodiment of the present disclosure;

FIG. 3 is a schematic structural diagram of a watch involved in a wearable device according to an embodiment of the present disclosure;

FIG. 4 is a schematic structural diagram of another watch involved in a wearable device according to an embodiment of the present disclosure;

FIG. 5 is an optional block diagram of a wearable device according to an embodiment of the present disclosure; and

FIG. 6 is a schematic flow diagram of a data processing method of a wearable device according to a preferred embodiment of the present disclosure.

Implementations of the object, functional features, and advantages of the present disclosure are further described with reference to the drawings in combination of embodiments.

### DETAILED DESCRIPTIONS

The technical solution in the embodiments of the present disclosure is clearly and completely described as following with reference to the drawings in the embodiments of the present disclosure.

It should be noted that, if there is a directional indication (such as up, down, left, right, front, back . . . ) involved in the embodiment of the present disclosure, the directional indication is only used to explain the relative positional relationship, motion condition, etc. between the components in a specific posture (as shown in the drawings), and if the specific posture changes, the directional indication also changes accordingly.

It should be noted that, if there are descriptions related to “first”, “second” etc. in the embodiments of the present disclosure, the descriptions of “first”, “second” etc. are merely intended for a purpose of description, and cannot be understood as indicating or implying relative importance or implicitly indicating a quantity of indicated technical features. Thus, features defining “first” and “second” may explicitly or implicitly include at least one of the features.

A first embodiment of the present disclosure provides a physiological sound collection apparatus including a bone conduction sensor and a first microphone, wherein the bone conduction sensor is configured to collect a physiological sound signal, the first microphone is configured to collect a first environmental noise signal, and the first environmental noise signal is used to correct the physiological sound signal.

The physiological sound signal includes heart sound signals, lung sound signals, etc. Among them, the heart sound refers to the sound generated by the mechanical wave phenomenon caused by myocardial contraction, heart valve closure, and blood hitting the ventricular wall, aortic wall, etc.; and the lung sound is mainly generated by the rupture of bubbles formed by gas passing through secretions of the respiratory and airway such as effusion, sputum, blood, etc. The heart sound signal is a signal obtained by acquiring the heart sound, and the lung sound signal is a signal obtained by acquiring the lung sound.

Taking collection of the heart sound signal as an example, the bone conduction sensor collects a vibration signal of the heart, and for the collected vibration signal, a filtering unit and a signal amplification unit are further required to eliminate the noise signal and amplify the wanted signal, and for the first environmental noise signal, processing of eliminating the noise signal and amplifying the wanted signal is also required. When the bone conduction sensor collects the heart sound signal, the user needs to put the earphone close to the chest wall, so that the heart sound signal collected by the bone conduction sensor is more accurate.

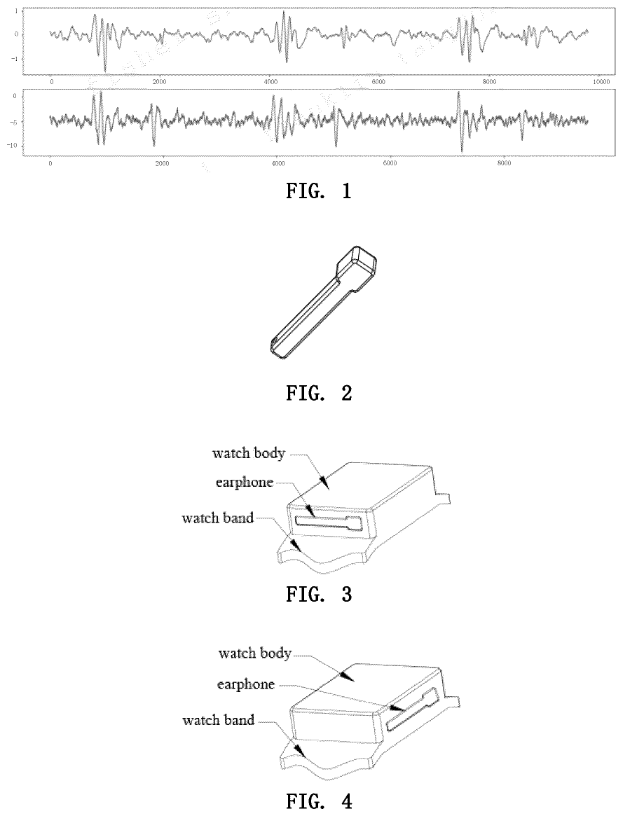

Referring to FIG. 1, FIG. 1 is a schematic diagram of a heart sound signal (upper) and a first environmental noise signal (lower) collected by a physiological sound collection apparatus of the present disclosure. For avoiding an collection error (including noise caused by a motion of a finger of the user, noise caused by friction between the earphone and clothes, etc.) caused by the physiological sound signal collection method, the heart sound signal needs to be corrected by using the first environmental noise signal collected by the first microphone. The specific process is as follows: extracting a key heart sound signal feature (determined by a normal heart sound signal when there is no lesion in the heart in medicine) as a mark point, and aligning the heart sound signal with the first environmental noise signal; and by taking the heart sound signal as a main signal and the first environmental noise signal as a correction signal, determining whether an abnormal signal (an abnormal sound feature) in the main signal is caused by an abnormal heart of the user or an collection error. Among them, the normal heart sound signal is used to indicate a normal signal (indicating a peak and/or a valley) in the heart sound signal, and the abnormal signal in the heart sound signal is a signal with an obvious peak and/or valley other than the normal signal. It should be noted that, when an abnormal signal exists in the heart sound signal and the abnormal signal also exists in the first environmental noise signal, it is determined that the abnormal signal is an collection error; and when an abnormal signal exists in the heart sound signal but the abnormal signal does not exist in the first environmental noise signal, it is determined that the abnormal signal is caused by the abnormal heart of the user.

As an example, the apparatus further includes a second microphone, wherein the second microphone is configured to collect a second environmental noise signal; and the environmental noise characteristics are removed from the physiological sound signal only when the second environmental noise signal contains the same environmental noise characteristics as that contained in the first environmental noise signal.

The second environmental noise signal also needs to be processed by eliminating the noise signal and amplifying the wanted signal. It should be noted that the position of the second microphone needs to be away from the bone conduction sensor, for example, both of them are arranged on opposite sides of the earphone, so that when the bone conduction sensor is close to the chest wall, the second microphone is away from the chest wall, so that the second microphone can receive clear environmental noise. The environmental noise is a sound that a person in the environment speaks loudly, a sound that a door is closed, etc. It should be noted that the noise reduction processing is a process of subtracting the second environmental noise signal from the heart sound signal.

It should be noted that, before noise reduction processing is performed on the heart sound signal by using the second environmental noise signal, the second environmental noise signal and the heart sound signal also need to be aligned.

The purpose of setting the second microphone is to perform secondary judgment on the abnormal signal in the heart sound signal, thereby improving the accuracy of removing the environmental noise characteristics in the heart sound signal.

As an example, the apparatus further includes a reminder module, wherein the reminder module is configured to output first reminder information to remind a user to adjust a measurement posture when a physiological sound signal intensity of the physiological sound signal is less than or equal to a preset physiological sound signal intensity threshold; and the reminder module is further configured to output second reminder information to remind the user to select a new environment to collect the physiological sound when a noise signal intensity of the second environmental noise signal is greater than or equal to a preset noise signal intensity threshold.

The reminding module includes a signal output module such as a speaker, a display, etc. configured to output a sound signal, a text signal or an image signal.

The reason why the physiological sound signal intensity of the physiological sound signal is less than or equal to the preset physiological sound signal intensity threshold is that the physiological sound collection apparatus is not close to the human body. For example, the user uses the physiological sound collection apparatus through clothing, or the physiological sound collection apparatus is not tightly fitting to the chest. In this case, the measurement posture of the user is incorrect, and the user is reminded by outputting the first reminder information to make the physiological sound collection apparatus close to the human body. For example, the first reminder information is “the physiological sound is not successfully collected, please try again after the apparatus is attached to the chest”. When the physiological sound signal intensity of the physiological sound signal is greater than the preset physiological sound signal intensity threshold, there is no need to output the reminder information. The preset physiological sound signal intensity threshold may be set as required, which is not specifically limited in this embodiment.

When the noise signal intensity of the second environmental noise signal is greater than or equal to the preset noise signal intensity threshold, the measured heart sound signal is seriously affected by noise, resulting in inaccurate collected physiological sound signal. Therefore, the user may be reminded to select a new environment to collect the physiological sound by outputting the second reminder information, and when the noise signal intensity of the second environmental noise signal is less than the preset noise signal intensity threshold, there is no need to output the second reminder information. For example, the second reminder information is “please perform physiological sound collection in a quiet environment”. The preset noise signal strength threshold may be set as required, which is not specifically limited in this embodiment.

Compared with a manner of acquiring physiological sound in which auscultation is performed when seeking medical attention, and the auscultation needs to be implemented by a doctor in a hospital, resulting in inconvenient collection of the physiological sound in the prior art, the present disclosure provides a physiological sound collection apparatus including a bone conduction sensor and a first microphone, wherein the bone conduction sensor is configured to collect a physiological sound signal, the first microphone is configured to collect a first environmental noise signal, and the first environmental noise signal is used to correct the physiological sound signal. In the present disclosure, the physiological sound signal is collected by the bone conduction sensor in the physiological sound collection apparatus, so that the physiological sound can be collected by the user of the apparatus without the operation of the doctor. In addition, after the physiological sound signal is corrected by using the first environmental noise signal collected by the first microphone, accuracy of the physiological sound signal is improved.

As an example, the physiological sound collection apparatus may be in a device such as an earphone or a watch, or may be an independent apparatus that has only a physiological sound collection function, and a specific implementation of the physiological sound collection apparatus is not specifically limited in this embodiment.

This embodiment takes the physiological sound collection apparatus in an earphone as an example. Referring to FIG. 2, FIG. 2 is a schematic structural diagram of an earphone involved in the physiological sound collection apparatus according to an embodiment of the present disclosure.

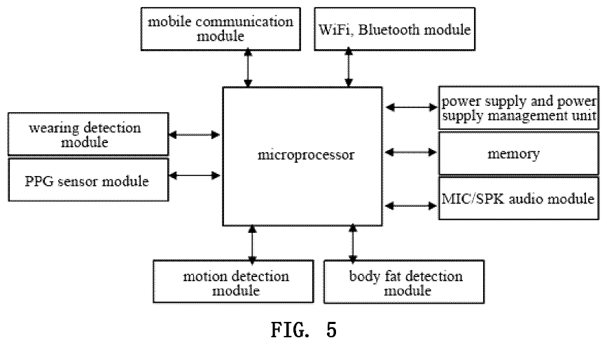

In a second embodiment of the present disclosure, the wearable device includes the above-mentioned physiological sound collection apparatus, the physiological sound collection apparatus is a physiological sound collection apparatus including a bone conduction sensor, a first microphone and a second microphone, or a physiological sound collection apparatus not including the second microphone. The wearable device may be a mobile phone, a tablet, a watch, a wristband, or the like. It should be noted that the physiological sound collection apparatus in the wearable device may be integrally disposed with the wearable device body, or may be separately disposed from the wearable device body. In a case of separate arrangement, the physiological sound collection apparatus may be wirelessly connected to the wearable device body, or may be wired connected to the wearable device body by using a connector (including a pogo-pin connector, an elastic piece connector, a wire connector, and the like). Optionally, in this embodiment, taking that the wearable device is a watch (including a physiological sound collection apparatus and a watch body), and the physiological sound collection apparatus is separately disposed and wirelessly connected to the watch body as an example, wherein the physiological sound collection apparatus is an earphone. Referring to FIG. 3 and FIG. 4, which are schematic diagrams of two structures of a watch involved in a wearable device according to an embodiment of the present disclosure. The earphone may be disposed on the watch in a manner such as embedding or magnetic attraction.

As an example, the wearable body includes a first communication module, and the earphone includes a second communication module;
the first communication module is configured to receive, from the second communication module, the physiological sound signal from which the environmental noise characteristics have been removed;
the first communication module includes a Bluetooth module or an Ultra Wide Band (UWB) module;
the second communication module includes a Bluetooth module or a UWB module.

It should be noted that the process of processing the heart sound signal in the first embodiment may also be implemented in the watch body, that is, the earphone sends the heart sound signal, the first environmental noise signal and the second environmental noise signal collected by the earphone to the watch body, and the watch body corrects and denoises the heart sound signal through the first environmental noise signal and the second environmental noise signal respectively. The correction processing and the noise reduction processing are basically the same as the implementation in the first embodiment, and details are not described herein again.

It can be understood that, when the physiological sound collection apparatus is integrated with the wearable device body, the user may take the wearable device to be close to the chest wall.

As an example, the wearable body further includes a heart monitoring trigger data collection module; the heart monitoring trigger data collection module is configured to collect heart monitoring trigger data; the heart monitoring trigger data is used to determine whether a user requires enhanced heart health monitoring, and when it is determined that the user requires enhanced heart health monitoring, the wearable body enters a heart monitoring mode.

As an example, the heart monitoring trigger data collection module is a body fat detection module, and the heart monitoring trigger data is a body fat rate; if the body fat rate is greater than or equal to a preset body fat rate threshold, it is determined that the user requires enhanced heart health monitoring, and the wearable body enters the heart monitoring mode.

The body fat detection module includes a Bio-impedance analysis (BIA) sensor.

It can be understood that, when the body fat rate of the user exceeds the standard, that is, the body fat rate is greater than or equal to the preset body fat rate threshold, the heart of the user is very likely to be in an unhealthy state, and it can be determined that such a user requires enhanced heart health monitoring; if the body fat rate is less than the preset body fat rate threshold, it may be determined that the heart of the user is unlikely in an unhealthy state, and the heart monitoring mode is not entered. The preset body fat rate threshold may be set as required, which is not specifically limited in this embodiment.

As an example, the heart monitoring trigger data collection module is a motion detection module, and the heart monitoring trigger data is motion data; the motion data is used to determine whether a frequency at which the user participates in exercise is less than or equal to a preset frequency threshold, and when it is determined that the frequency at which the user participates in exercise is less than or equal to the preset frequency threshold, it is determined that the user requires enhanced heart health monitoring, and the wearable body enters the heart monitoring mode. The motion detection module includes an A+G motion sensor.

The motion data includes running record data, basketball playing record data, badminton playing record data, and the like. For example, if there is a running record on August 5, a running record on September 1, and a running record on October 3, it is determined that the running frequency of the user is once a month, and the motion frequency of the user is once a month. For another example, if there is a running record on August 5, a basketball playing record on August 20, a running record on September 1, a basketball playing record on September 23, a running record on October 3, a badminton playing record on October 18, and a basketball playing record on November 4, the motion frequency of the user is twice a month.

It should be noted that the preset frequency threshold may be set as required, which is not specifically limited in this embodiment.

As an example, in order to improve the accuracy of determining whether the user requires enhanced heart health monitoring, the determination may be performed by combining the data acquired by the body fat detection module and the data acquire by the motion detection module. Specifically, the heart monitoring trigger data collection module includes a body fat detection module and a motion detection module, and the heart monitoring trigger data includes a body fat rate and motion data; and if the body fat rate is greater than or equal to a preset body fat rate threshold and a frequency at which the user participates in exercise is less than or equal to a preset frequency threshold, it is determined that the user requires enhanced heart health monitoring, and the wearable body enters the heart monitoring mode.

It can be understood that, in this embodiment, it is determined that the user requires enhanced heart health monitoring only when the body fat rate is greater than or equal to the preset body fat rate threshold and the frequency at which the user participates in exercise is less than or equal to the preset frequency threshold, and it is determined that the user does not require enhanced heart health monitoring when the body fat rate is greater than or equal to the preset body fat rate threshold or the frequency at which the user participates in exercise is less than or equal to the preset frequency threshold.

This embodiment avoids misjudgment on whether the user requires enhanced heart health monitoring, and improves the accuracy of determining whether the user requires enhanced heart health monitoring. That is, when the body fat rate is greater than or equal to the preset body fat rate threshold, but the user is aware of the issue and has started to exercise frequently; or when the frequency at which the user participates in exercise is less than or equal to the preset frequency threshold, but the body fat rate is less than the preset body fat rate threshold, the user's heart may not be prone to problems.

As an example, the wearable body further includes a wearing detection module and a physiological parameter signal collection module;
the wearing detection module is configured to determine whether the wearable body is in a wearing state; and
the physiological parameter signal collection module is configured to collect a physiological parameter signal of the user when the wearable body is in the wearing state.

In order to avoid collecting the physiological parameter signal when the user is not wearing the wearable body and thus increasing the power consumption of the wearable body and reducing the endurance of the wearable body, in this embodiment, whether the wearable body is in the wearing state is determined by the wearing detection module, so as to improve the endurance of the wearable body. The wearing detection module includes a capacitance (CAP) sensor, an infrared sensor, an ultrasonic sensor, a heart rate sensor, and the like.

As an example, the wearable body further includes a photoplethysmography (PPG) sensor module, and the PPG sensor module is configured to collect a physiological parameter signal of the user.

Here, the PPG sensor module includes a PPG sensor and a signal amplification unit, and the physiological parameter signal collected by the PPG sensor also needs to be amplified by the signal amplification unit.

After the wearable body enters the heart monitoring mode, the PPG sensor module monitors indexes such as heart rate and blood oxygen (which are implemented by the collected physiological parameter signals).

As an example, the wearable body further includes a reminding module, and the reminding module is configured to provide health reminders to the user according to the health risk level corresponding to the physiological parameter signal.

The health risk level is set according to how much the index exceeds the normal index, and the health risk level may be levels of high, medium, low, levels of high, medium, lower, low, or the like. Taking the health risk level being the levels of high, medium, low as an example, how much the index exceeds the normal index correspond to the levels of high, medium, low of the health risk level. For example, the index is a heart rate index, the normal heart rate index is 100, when the heart rate is greater than 100 and less than 120, it is determined that the health risk level is low, and the user may not be reminded at this time; when the heart rate is greater than or equal to 120 and less than 160, it is determined that the health risk level is medium, and the user may be recommended to detect the heart sound at this time; and when the heart rate is greater than or equal to 160, it is determined that the health risk level is high, and the user is recommended to rest or seek medical attention immediately at this time.

The reminding module includes a vibration unit, a sounding unit, etc., and the user may be reminded by vibrating the vibration unit and/or sounding the sounding unit when reminding.

It should be noted that relevant data (electrocardiogram data obtained by processing the physiological parameter signal) may also be uploaded to the mobile phone through a wireless communication module (including a WIFI/Bluetooth module, etc.), so that the corresponding APP in the mobile phone draws a heart health graph according to the relevant data, and gives relevant life and medical advices.

It should be noted that the PPG sensor module and the body fat detection module are both disposed on a side in direct contact with the user's skin to accurately collect related signals. The number thereof is not specifically limited in this embodiment, and may be set as required.

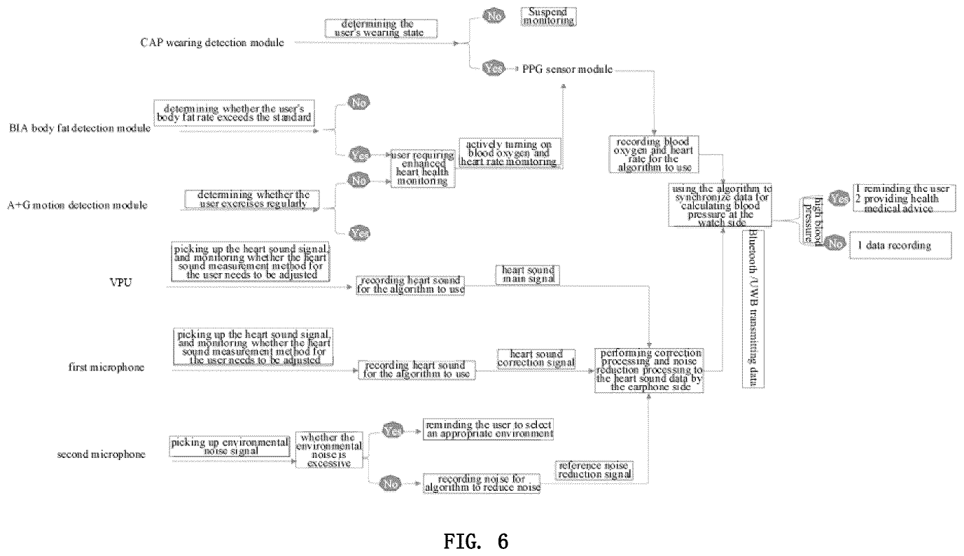

As an example, the wearable device may further include other units or modules. Referring to FIG. 5 and FIG. 6, FIG. 5 is an optional block diagram of a wearable device according to an embodiment of the present disclosure, and FIG. 6 is a schematic flow diagram of a data processing method of a wearable device according to a preferred embodiment of the present disclosure. Among them, a microprocessor can achieve operations such as fetching instructions, executing instructions, as well as exchanging information with external memory and logic components. The microprocessor is an operation control part of the wearable device, for example, it is configured to process the heart sound signal, the first environmental noise signal, the physiological parameter signal, and the like. A mobile communication module includes a 4G mobile communication module, a 5G mobile communication module, and the like, so that the wearable device can be connected to an external network (for example, the Internet) by using the module. A memory is configured to store various application programs and related data (the heart sound signal, the first environmental noise signal, the physiological parameter signal, and the like). A power supply and a power supply management unit are configured to supply power to the wearable device and perform power consumption monitoring, power consumption control, and the like. An audio module, in addition to collecting a sound signal (for example, the heart sound signal), may further include an audio decoding unit, an audio operational amplifier unit, an MIC unit, a speaker unit, etc., which may decode audio content and generate sound.

As an example, the wearable body further includes a processor;
the processor is configured to calculate a blood pressure of the user based on the physiological sound signal from which the environmental noise characteristics have been removed and the physiological parameter signal, and perform a corresponding device operation based on the blood pressure.

If the blood pressure is high blood pressure, the device operation includes reminding the user of the high blood pressure through the reminding module or providing health medical advice through the reminding module. If the blood pressure is not high blood pressure, the device operation is recording data for the user to review or as the basis of relevant statistical data.

It should be noted that, in addition to calculating the blood pressure by using the data, the wearable body may further determine, by using related data, whether the user has a premature heartbeat.

It should be noted that the above are only preferred embodiments of the present disclosure, and are not intended to limit the patent scope of the present disclosure. Any equivalent structural transformation made by using the content of the specification and the accompanying drawings of the present disclosure or direct/indirect applications in other related technical fields, under the inventive concept of the present disclosure, are included in the patent protection scope of the present disclosure.

1. A physiological sound collection apparatus, comprising:
a bone conduction sensor and a first microphone,
wherein the bone conduction sensor is configured to collect a physiological sound signal, the first microphone is configured to collect a first environmental noise signal, and the first environmental noise signal is used to remove environmental noise characteristics from the physiological sound signal.

2. The apparatus according to claim 1, further comprising a second microphone configured to collect a second environmental noise signal,
wherein the environmental noise characteristics are removed from the physiological sound signal only when the second environmental noise signal contains the same environmental noise characteristics as that contained in the first environmental noise signal.

3. The apparatus according to claim 2, further comprising a reminder module configured to:
output first reminder information to remind a user to adjust a measurement posture when a physiological sound signal intensity of the physiological sound signal is less than or equal to a preset physiological sound signal intensity threshold; and
output second reminder information to remind the user to select a new environment to collect the physiological sound when a noise signal intensity of the second environmental noise signal is greater than or equal to a preset noise signal intensity threshold.

4. A wearable device, comprising:
a wearable body and an earphone,
wherein the earphone comprises the physiological sound collection apparatus according to claim 1.

5. The wearable device according to claim 4, wherein the wearable body comprises a first communication module, and the earphone comprises a second communication module,
wherein the first communication module is configured to receive, from the second communication module, the physiological sound signal from which the environmental noise characteristics have been removed,
wherein the first communication module comprises a Bluetooth module or a UWB module, and
wherein the second communication module comprises a Bluetooth module or a UWB module.

6. The wearable device according to claim 5, wherein the wearable body further comprises a heart monitoring trigger data collection module configured to collect heart monitoring trigger data,
wherein the heart monitoring trigger data is used to determine whether a user requires enhanced heart health monitoring, and when it is determined that the user requires enhanced heart health monitoring, the wearable body enters a heart monitoring mode.

7. The wearable device according to claim 6, wherein the heart monitoring trigger data collection module is a body fat detection module, and the heart monitoring trigger data is a body fat rate, and
wherein if the body fat rate is greater than or equal to a preset body fat rate threshold, it is determined that the user requires enhanced heart health monitoring, the wearable body enters the heart monitoring mode.

8. The wearable device according to claim 6, wherein the heart monitoring trigger data collection module is a motion detection module, and the heart monitoring trigger data is motion data, and
wherein the motion data is used to determine whether a frequency at which the user participates in exercise is less than or equal to a preset frequency threshold, and when it is determined that the frequency at which the user participates in exercise is less than or equal to the preset frequency threshold, it is determined that that the user requires enhanced heart health monitoring, and the wearable body enters the heart monitoring mode.

9. The wearable device according to claim 6, wherein the heart monitoring trigger data collection module comprises a body fat detection module and a motion detection module, and the heart monitoring trigger data comprises a body fat rate and motion data, and
wherein if the body fat rate is greater than or equal to a preset body fat rate threshold and a frequency at which the user participates in exercise is less than or equal to a preset frequency threshold, it is determined that the user requires enhanced heart health monitoring, and the wearable body enters the heart monitoring mode.

10. The wearable device according to claim 6, wherein the wearable body further comprises a wearing detection module and a physiological parameter signal collection module,
wherein the wearing detection module is configured to determine whether the wearable body is in a wearing state, and
wherein the physiological parameter signal collection module is configured to collect a physiological parameter signal of the user when the wearable body is in the wearing state.

11. The wearable device according to claim 10, wherein the wearable body further comprises a processor configured to calculate a blood pressure of the user based on the physiological sound signal from which the environmental noise characteristics have been removed and the physiological parameter signal, and perform a corresponding operation based on the calculated blood pressure.
